(19) United States
(12) Patent Application Publication
(10) Pub. No.: US 2008/0075660 A1
(43) Pub. Date: Mar. 27, 2008

(54) Near infrared-fluorescence using phospholipid ether analog dyes in endoscopic applications

(75) Inventors: Jamey Weichert, Fitchburg, WI (US); Marc Longino, Verona, WI (US); Anatoly Pinchuk, Madison, WI (US); Andreas Muehler, Mountain Lakes, NJ (US)

Correspondence Address: WOOD, PHILLIPS, KATZ, CLARK & MORTIMER, 500 W. MADISON STREET, SUITE 3800, CHICAGO, IL, 60661, US

(73) Assignee: Cellectar, LLC, Madison, WI (US)

(21) Appl. No.: 11/891,939

(22) Filed: Aug. 14, 2007

Related U.S. Application Data
(60) Provisional application No. 60/822,448, filed on Aug. 15, 2006.

(51) Int. Cl. A61K 51/04; A61B 8/12; A61P 43/00

(52) U.S. Cl. 424/1.77; 424/9.6

(57) ABSTRACT

The present invention provides composition and methods of use of phospholipid dyes for use in detection of neoplastic tissue, typically using the routing procedure of endoscopy and methods of optimizing therapy treatment in a subject.

### RELATED FIELD

The invention generally relates to phospholipid ether (PLE) analogs for diagnosis of neoplasia, in particular, the Invention relates to use of phospholipid ether dyes in endoscopic application using near infrared fluorescence.

### BACKGROUND

Endoscopy, in particular colonoscopy and bronchoscopy, is utilized to find abnormal growth and tumors protruding into the lumen. A device, called endoscope, is inserted into a body cavity. Traditionally, endoscopes use a daylight channel, i.e. the observer sees all finding at the wavelength of naturally occurring light.

Lately, newer endoscopes have the ability to utilize several channels, i.e. using a daylight channel and one or more additional channels at other light wavelengths. These additional channels are used to monitor either naturally occurring fluorescence or fluorescence of a dye that was either injected into the body or sprayed onto the body cavity surface. One of the possible channels is in the NIR (near Infrared) area. The advantage of the NIR area Is that the light absorption in the NIR area (usually 600-800 nm) is minimal, and fluorescence can be detected at a depth of a few millimeters to nearly a centimeter beneath the surface of the body cavity. It is believed that this has advantages to detect tumors and lymph node metastases in organs such as colon and lung.

Accordingly, the need exists to further explore the uses of near infrared fluorescence in detecting neoplasia during the endoscopic process.

### SUMMARY OF THE INVENTION

The invention generally relates to phospholipids ether (PLE) analogs for diagnosis of neoplasia, in particular, the invention relates to use of phospholipid ether dyes in endoscopic application using near infrared fluorescence. In an exemplary embodiment, the present invention provides a phospholipid fluorescent dye, comprising (a) a phospholipid compound of formula I or II

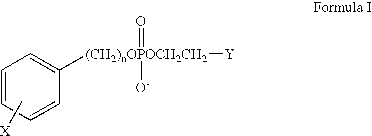

where X is a halogen; n is an integer between 8 and 30; and Y is selected from the group comprising NH₂, NR₂, and NR₃, wherein R is an alkyl or arylalkyl substituent or

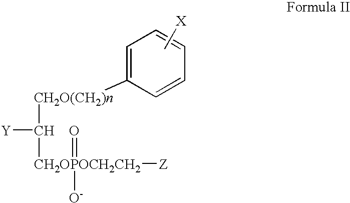

where X is a halogen; n is an integer between 8 and 30; Y is selected from the group consisting of H, OH, COOH, COOR and OR, and Z is selected from the group consisting of NH₂, NR₂, and NR₃, wherein R is an alkyl or arylalkyl substituent; and (b) a fluorescent molecule. In this embodiment, X is selected from the group of radioactive halogen Isotopes consisting of ¹⁸F, ³⁶Cl, ⁷⁶Br, ⁷⁷Br, ⁸²Br, ¹²²I, ¹²³I, ¹²⁴I, ¹²⁵I, ¹³¹I and ²¹¹At. Preferably, the phospholipid compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive isotope. In yet another exemplary embodiment, the phospholipid dye is selected from the group consisting of 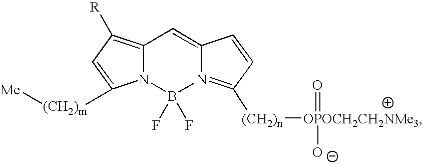
wherein n is an integer 4 through 21 and m is an integer 0 through 17; 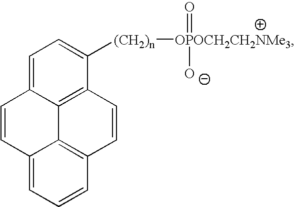
wherein n is an integer 4 through 22; 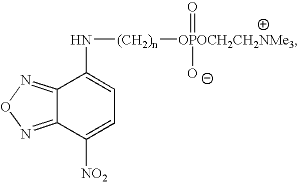
wherein n is an integer 4 through 22; 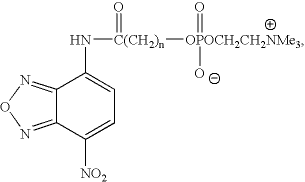
wherein n is an integer 4 through 21; 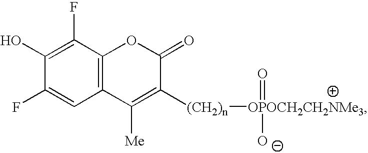
wherein n is an integer 4 through 22; 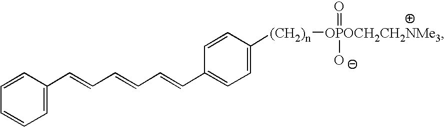
wherein n is an integer 3 through 8; and 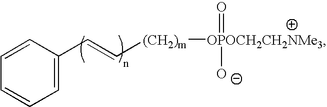
wherein n is an integer 4 or 5 and m is an integer 4 through 14.

Further, in this embodiment, the fluorescent molecule exhibits fluorescence at a wavelength of about 300 nm to about 1000 nm.

Another exemplary embodiment of the invention provides a method for distinguishing a benign structure from a neoplastic tissue in a selected region by using an endoscope have at least two wavelength in a subject comprising the steps of: (a) administering a fluorescently labeled tumor-specific agent to the subject; (b) using a first technique to produce a visualization of the anatomy of the selected region using the first wavelength of an endoscope; (c) using a second technique to produce a visualization of the distribution of fluorescence produced by the fluorescently labeled tumor-specific agent; and (d) comparing the visualization of the anatomy of the selected region by the first wavelength to the visualization of the distribution of fluorescence by the second wavelength produced by the fluorescently labeled tumor-specific agent thereby distinguishing a benign structure from neoplastic tissue. In this embodiment, preferably, the selected region is the gastro-intestinal tract and the respiratory tract.

In this embodiment, the first wavelength is about 400 nm to about 800 nm. Also, the second wavelength is about 300 nm to 1000 nm.

Preferably, the fluorescently labeled tumor selective compound is a phospholipid dye, comprising of (a) a phospholipid compound of formula I or II

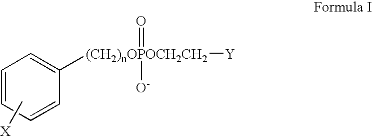

where X is a halogen; n is an integer between 8 and 30; and Y is selected from the group comprising NH₂, NR₂, and NR₃, wherein R is an alkyl or arylalkyl substituent or

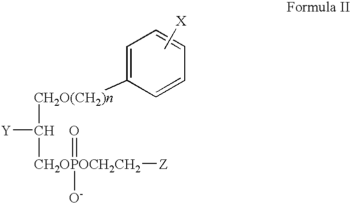

where X is a halogen; n is an integer between 8 and 30; Y is selected from the group consisting of H, OH, COOH, COOR and OR, and Z is selected from the group consisting of NH₂, NR₂, and NR₃, wherein R is an alkyl or arylalkyl substituent; and (b) a fluorescent molecule. Further, X is selected from the group of radioactive halogen isotopes consisting of ¹⁸F, ³⁶Cl, ⁷⁶Br, ⁷⁷Br, ⁸²Br, ¹²²I, ¹²³I, ¹²⁴I, ¹²⁵I, ¹³¹I and ²¹¹At.

Most preferably, the phospholipid compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive isotope. Also, preferably, the dye is selected from the group consisting of 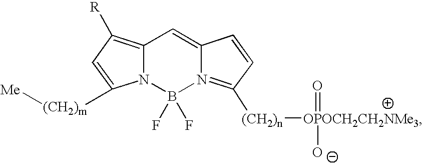
wherein n is an Integer 4 through 21 and m is an integer 0 through 17; 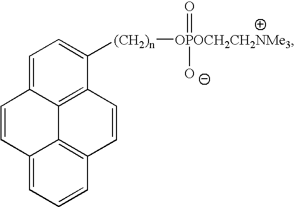
wherein n is an integer 4 through 22; 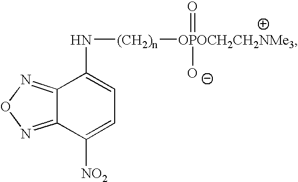
wherein n is an integer 4 through 22; 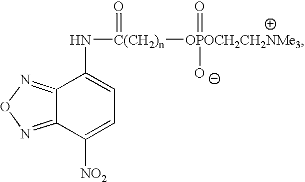
wherein n is an integer 4 through 21; 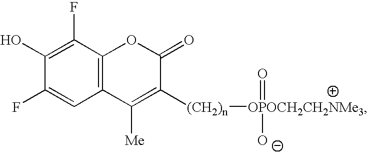
wherein n is an integer 4 through 22; 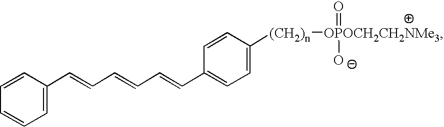
wherein n is an integer 3 through 8; and 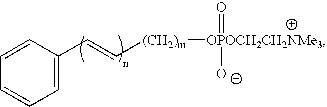
wherein n is an integer 4 or 5 and m is an integer 4 through 14.

Further, in this method, the fluorescent molecule exhibits fluorescence at a wavelength of about 300 nm to about 1000 nm.

In yet another embodiment, the present invention provides a method of optimizing therapy treatment in a subject, comprising the steps of: (a) providing a radiolabeled phospholipid compound wherein said compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive isotope, in a quantity of about 1 millicurie to about 100 millicurie; (b) visualizing neoplastic tissue via SPECT or PET imaging; (c) assessing therapy dosage to the subject by quantifying the distribution of the neoplastic tissue.

Another embodiment of the invention provides a method of monitoring tumor therapy response in a subject or effectiveness of a treatment methodology in a subject receiving the treatment for neoplasia, comprising the steps of: (a) providing a radiolabeled phospholipid compound to the subject prior to treatment of neoplasia wherein said compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive isotope, in a quantity of about 1 mililcurie to about 100 millicurie; (b) providing the radiolabeled phospholipid compound to the subject of step (a), after the treatment of neoplasia in a quantity of about 1 millicurie to about 100 millicurie; and (c) assessing difference in accumulation of the phospholipid compound from the pre-treatment of step (a) and the post-treatment of step (b) to determine the response in a subject or effectiveness of the treatment methodology, wherein a greater accumulation of the phospholipid compound in step (a) versus lesser accumulation of phospholipid compound In step (b) indicates a positive response to the treatment in a subject or an effective treatment methodology.

### FIGURES

FIG. 1 provides a 2D microCT projection of an excised PIRC rat colon filled with 2% barium (A) and ¹²⁴I-NM404 microPET image in a PIRC Rat (B) and the fused microPET/microCT image (C). Fiducial marker (M), Tumor (arrow).

### DETAILED DESCRIPTION OF PREFERRED EMBODIMENTS

The phospholipid ether analogs that can be used for imaging various tumors are defined by formula I and II: wherein in formula II X is a radioactive Isotope of a halogen, n is an integer between 8 and 30, Y is selected from the group consisting of H, OH, COOH, O(CO)R, and OR, and Z is selected from the group consisting of NH₂, NR₂, and NR₃, wherein R is an alkyl or aralkyl substituent; and wherein in formula IIX is a radioactive isotope of a halogen, n is an integer between 8 and 30, and Y is selected from the group comprising NH₂, NR₂, and NR₃, wherein R is an alkyl or aralkyl substituent.

NM404 and other PLE-based compounds have been known from studies of radiolabeled versions (such as 1-124) that these compounds accumulate in malignant tumors, but not in benign tumors such as polyps. An example is given below that the accumulation of NM404 can be used to differentiate benign and malignant tumors. Various PLE-based compounds, such as those described below are also described in various other patents and patent applications. See U.S. provisional applications 60/521,166 filed on Mar. 2, 2005, 60/521,831 filed in Jul. 8, 2005, 60/593,190 filed on Dec. 20, 2004 and 60/743,232 filed on Feb. 3, 2006; U.S. non-provisional application Ser. Nos. 10/906,687 filed on Mar. 2, 2005, 11/177,749 filed on Jul. 8, 2005 and 11/316,620 filed on Dec. 20, 2005, PCT Applications PCT/US05/006681 filed on Mar. 2, 2005, PCT/US05/024259 filed on Jul. 8, 2005 and PCT/US05/047657 filed on Dec. 20, 2005; U.S. Pat. Nos. 4,925,649, 4,965,391, 5,087,721, 5,347,030, 5,795,561, 6,255,519 and 6,417,384; Patent publications WO1998/024480 and WO1998/024480; and Canadian Application 2,276,284, all of which are incorporated by reference, as though fully set forth herein.

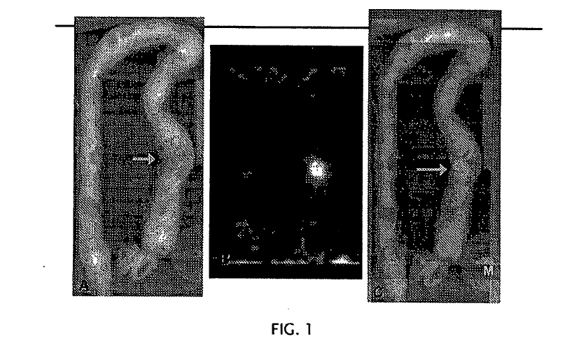

As depicted in FIG. 1, the left image shows an ex-vivo microCT Image of a colon tumor model in rats. Multiple tumors have been detected protruding into the colon lumen. The middle image shows a microPET image using I-124-NM404 of the same colon showing one area of accumulation only. The right image shows a fusion image of MicroCT/microPET that confirms that the accumulation of NM404 was seen only in a tumor that later proved to be an adenocarcinoma. All other colon tumors turned out to be benign polyps and such did not show accumulation of NM404.

It was also previously shown that PLE compounds like NM404 can be labeled with bulky signaling moieties such as fluorescent dyes. See for example, Delgado et al, Fluorescent phenylpolyene analogues of the ether phospholipid edelfosine for the selective labeling of cancer cells, J Med. Chem. 2004, 47(22):5333-5.

Numerous fluorescent tags are known to one of skill in the art. Methodologies for tagging PLE compounds such as NM404 with fluorescent dyes are also known in the art. Once the PLE compound tagged with a fluorescent dye is prepared by known methodologies, in one exemplary embodiment, the invention describes the use of such PLE compounds such as NM404 labeled with NIR fluorescent moieties (called NIR-PLE dyes). Such NIR-PLE dye is injected intravenously a few hours before performing endoscopic examinations. An endoscope with at least a daylight and NIR channel is used to examine the body cavity. In operation, the physician may switch between both daylight and NIR channels. The daylight channel Is used to detect any abnormal growth or tumors. When those are found, the physician may switch to the NIR channel to determine whether such growth or tumors is malignant or benign. These information can be used for three indications: 1) to diagnose the growth or tumor, 2) to identify the best and most optimal area for a biopsy, or c) to immediately remove (resect) such growth or tumor via minimal surgical methods. Body cavities that the inventions can be used in include, but are not limited to colon, rectum, bronchi, lung, sinus, pancreatic or biliary duct, esophagus, stomach, duodenum, uterus and intra-abdominal cavity.

Fluorescent analogs of NM404

In a exemplary embodiment, several fluorescent analogs of NM404 are provided which may be used as probes as described above. These probes bear structural resemblance to NM404. The fluorophores in these probes are incorporated into hydrophobic alkyl chain of NM404.

In an exemplary embodiment, BODIPY^{⊕} (500 nm/510 nm) analogs may be used in which the green-fluorescent fluorophores are located within the alkyl chain of NM404:

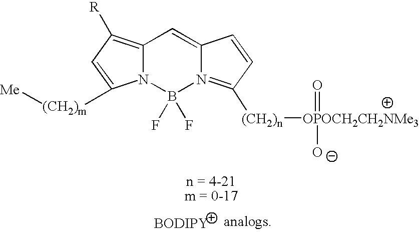

In another exemplary embodiment, pyrene analogs (344 nm/378 nm) may be used having 4 to 22 carbons in the alkyl chain:

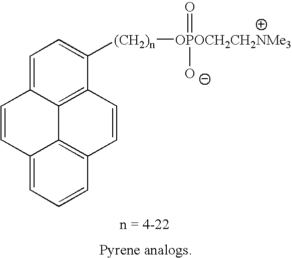

In yet another exemplary embodiment, NBD (nitrobenzoxadiazole) analogs (463 nm/536 nm) may be used in which fluorophore is attached either via amine or amide bond

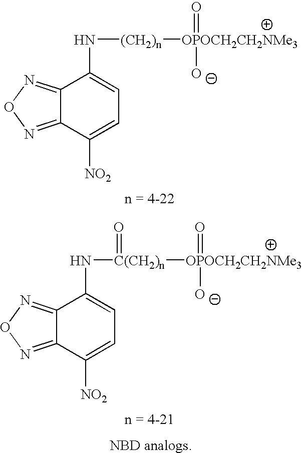

In another exemplary embodiment, Coumarin analogs may be used. One example shown below has Marina Blue, (6,8-difluoro-7-hydroxycoumarin) fluorophore (365 nm/460 nm) with 4 to 22 methylene groups:

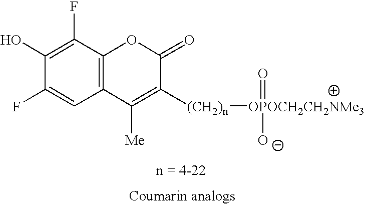

Yet other analogs containing DPH (diphenylhexatriene) fluorophore (350 nm/452 m) may be used:

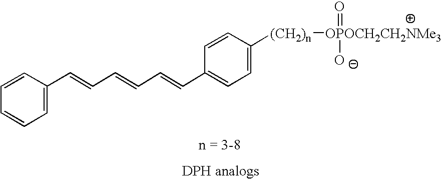

In another exemplary embodiment, group of analogs bearing polyene fluorophore may be used. Fluorophore with n=4 and m=7 was described in J Med. Chem. 2004; 47 (22): 5333-5 being incorporated into ET-18-OCH₃ analog.

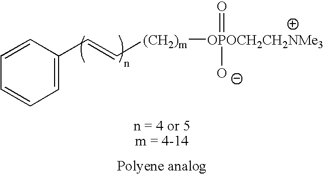

Other examples and methodologies for synthesizing fluorescent probes are provided in O. Maier et al. Fluorescent lipid probes: some properties and applications (a review), Chem. Phys. Lipids, 2002; 116(1):3-18.

In yet another exemplary embodiment, PLE compounds may be used for tumor therapy response monitoring. Previously, NM404 and other PLE-based compounds were shown to enter and be selectively retained in viable malignant cells. However, cells with impaired status such as those undergoing necrosis were shown to lack significant accumulation of NM404 or other PLE-based compounds. In one exemplary embodiment, the invention provides that this differential property of accumulation in viable and impaired malignant cells can be used to monitor therapy response. Tumor treatments aim to Impair the viability of malignant cells in many ways. If an examination with NM404 (or other PLE-based compounds) is performed before and following therapy, the potential difference in the accumulation of the compound Is due to the impairment of metabolism of cancer cells. If no such difference is found, the therapy has to be regarded non-effective. If a significant drop of accumulation between pre- and post-therapy is found, then the therapy has achieved its goal. The monitoring should Ideally be performed with a radioactively labeled PLE compound to be monitored by SPECT or PET Imaging, however also fluorescent or NIR methods can be used. This methodology may be useful for measuring not only the response of tumor therapy on a subject, but may also be useful for measuring effectiveness of any treatment methodology in the subject, such as radiation or chemotherapy using PLE or other cancer therapeutic agents.

In yet another exemplary embodiment, PLE compounds may be used in treatment planning for patients receiving the NM404 treatment. NM404 and other PLE-based compounds have been shown to be effective tumor therapies following intravenous injection. However, the effectiveness and effective dose level is known to depend on tumor uptake characteristics, tumor location, tumor perfusion, tumor viability and tumors size. It is difficult to individualize the treatment and Inject the most optimal dose with such factors unknown. Nuclear medicine methods like PET or SPECT allow quantitative or at least semi-quantitative assessment of concentration of radioactive tracers. This Information can be used to calculate the accumulation of an injected radioactive compound. The Invention provides that a tracer dose of radioactive compound such as NM404 or other PLE-based compound may be given to a subject. Such tracer dose (e.g. less than 10 mCi per patient, labels could be 1-124 for PET or 1-131 for SPECT) determines the individual accumulation characteristics for the tumor to be treated later on with a therapeutic dose of NM404 or another PLE-based compound. Based on these quantitative findings using the “trace dose”, the “treatment dose” can be individualized for each patient and treatment.

Typically, radionucilde therapy extends the usefulness of radiation from localized disease to multifocal disease by combining radionuclides with disease-seeking drugs, such as antibodies or custom-designed synthetic agents. DeNardo et al., Cancer Biotherapy & Radiopharmaceuticals, 2002, 17(1): 107-118. Like conventional radiotherapy, the effectiveness of targeted radlonuclides is ultimately limited by the amount of undesired radiation given to a critical, dose-limiting normal tissue, most often the bone marrow. Because radionuclide therapy relies on biological delivery of radiation, its optimization and characterization are necessarily different than for conventional radiation therapy. However, the principals of radiobiology and of absorbed radiation dose remain important for predicting radiation effects. Fortunately, most radionuclides emit gamma rays that allow the measurement of isotope concentrations in both tumor and normal tissues in the body. By administering a small “test dose” of the intended therapeutic drug, the clinician can predict the radiation dose distribution in the patient. This can serve as a basis to predict therapy effectiveness, optimize drug selection, and select the appropriate drug dose, in order to provide the safest, most effective treatment for each patient. Although treatment planning for individual patients based upon tracer radiation dosimetry is an attractive concept and opportunity, practical considerations may dictate simpler solutions under some circumstances. There is agreement that radiation dosimetry (radiation absorbed dose distribution, cGy) should be utilized to establish the safety of a specific radionuclide drug during drug development, but it is less generally accepted that absorbed radiation dose should be used to determine the dose of radionuclide (radioactivity, GBq) to be administered to a specific patient (i.e., radiation dose-based therapy). However, radiation dosimetry can always be utilized as a tool for developing drugs, assessing clinical results, and establishing the safety of a specific radionuclide drug. Bone marrow dosimetry continues to be a “work in progress.” Blood-derived and/or body-derived marrow dosimetry may be acceptable under specific conditions but clearly do not account for marrow and skeletal targeting of radionuclide. Marrow dosimetry can be expected to improve significantly but no method for marrow dosimetry seems likely to account for decreased bone marrow reserve.

Various dosimetry determinations may enable a physician to Inject a dose or find the individualization of treatment regimen that will provide the most effective treatment regimen (e.g. fractionated dosing) with an optimal treatment effect that produces the least side effects. Such assessment will likely Involve a dedicated software to be used to individualize treatment planning.

Radioiodination of NM404 in Preparation for Clinical Use (Prophetic)

A 2-ml glass vial is charged with 10 mg of ammonium sulfate dissolved in 50 μl of deionized water. Six 2 mm glass beads are added, then a Teflon-lined septum and screw cap are added and the vial gently swirled. A solution of 20 μg (in 20 μl of ethanol) of stock NM404 is added followed by aqueous sodium iodide (e.g., 125, 131, or 124, 1-5 mCi) in less than 30 μl aqueous 0.01 N sodium hydroxide. The isotope syringe is rinsed with three 20 μl portions of ethanol. The resulting reaction vial is swirled gently. A 5-ml disposable syringe containing glass wool in tandem with another 5-ml charcoal nugget filled syringe with needle outlet are attached. The glass wool syringe acts as a condensation chamber to catch evaporating solvents and the charcoal syringe traps free iodide/iodine. The resulting reaction vessel is heated in a heating block apparatus for 45 minutes at 150° C. Four 20 ml volumes of air are injected into the reaction vial with a 25-ml disposable syringe and allowed to vent through the dual trap attachment. The temperature is raised to 160° C. and the reaction vial heated another 30 minutes. After cooling to room temperature, ethanol (200 μl) is added and the vial swirled. The ethanolic solution is then passed through a pre-equilibrated Amberlite IRA 400 resin column to remove unreacted iodide. The eluent volume is reduced to 50 μl via a nitrogen stream (use charcoal syringe trap) and the remaining volume injected onto a silica gel column (Perkin Elmer, 3 μm×3 cm disposable cartridge column eluted at 1 ml/min with hexane/isopropanol/water (52:40:8)) for purification. Final purity is determined by TLC (plastic backed silica gel-60 eluted with chloroform-methanol-water (65:35:4, Rf=0.1). The HPLC solvents are removed by rotary evaporation and the resulting radioiodinated NM404 solubilized n aqueous 2% Polysorbate-20 and passed through a 0.22 μm filter into a sterile vial.

¹²⁴I-NM404-PET Imaging in Patients (Prophetic)

¹²⁴I-NM404 maximum dose for human administration is calculated as follows: Animal biodistribution data is generated to determine the percentage of injected dose/organ at varying time points. These animal data are extrapolated to man by means of MIRD formalism (MIRDOSE PC v3.1) using standard conversion factors for differences in organ mass and anatomy between rat and standard man, providing predicted human organ doses. Based on these predicted doses, the permissible mCi dose to be injected into humans is determined using the maximal doses legally permitted by RDRC regulations for specific human tissue as defined in the Federal Register (21CFR Part 361.1). For example, based on the ¹³¹I-NM404 data it is expected that the maximum starting dosage for 1241-NM404 should be below 2.0 mCi for pancreatic tumor imaging.

Patients receive SSKI (2 drops three times daily beginning 1 day before and continuing for seven days) in order to minimize uptake of free radioiodide by the thyroid. Patients allergic to iodine may be given potassium perchlorate (200 mg every 8 hours) starting one day before injection and continuing for 3 days post injection. 1241-NM404 is administered intravenously over 5 minutes. A transmission scan using a Ga-68/Ge-68 rotating positron emitting pin source is performed to measure the attenuation. These data are used for attenuation correction of emission data.

The patients are scanned at one or more of the following multiple timepoints following infusion of the 124I-NM-404: 90 minutes dynamic acquisition, 6 hours, 24 hours, 48 hours, and 96 hours.

The PET images are acquired in 2D mode with a BGO based GE ADVANCE PET scanner with an axial field of view of 152 mm. The images are acquired in 256×256 matrix and reconstruction is performed using a Hanning filter. All the images are attenuation corrected using the transmission data.

Before infusion, an Intravenous line is established in the upper extremity. The 1241-NM404 dose is measured in a dose calibrator prior to injection. A tracer dose of <2 mCi of ¹²⁴I-NM04 is infused over 2-5 minutes. The preparation is sterile, pyrogen-free, and contains <5% free iodine by thin layer chromatography (usual syntheses yield free radioiodine of about 1%).

Phantom studies using 1241 are performed to determine the calibration factor for the PET scanner and well counter. Phantom studies are performed for the same imaging times and same duration of acquisition.

The influx constant of the target region of uptake for any given patient is compared to a background region in the same patient and the lesions are classified as tumor or non-tumor regions based on this comparison. Similar classification of tumor and non-tumor region can also be done by visual analysis.

The present invention is not intended to be limited to the foregoing examples, but encompasses all such modifications and variations as come within the scope of the appended claims.

What is claimed is:

1. A phospholipid fluorescent dye, comprising
(a) a phospholipid compound of formula I or II 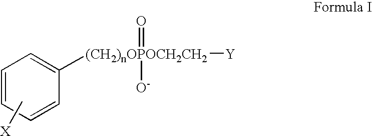
where X is a halogen; n is an integer between 8 and 30; and Y is selected from the group comprising NH₂, NR₂, and NR₃, wherein R is an alkyl or arylalkyl substituent or 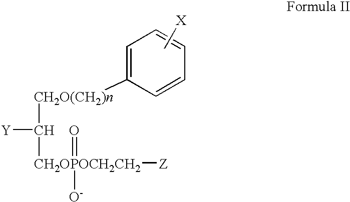
where X is a halogen; n is an integer between 8 and 30; Y is selected from the group consisting of H, OH, COOH, COOR and OR, and Z is selected from the group consisting of NH₂, NR₂, and NR₃, wherein R is an alkyl or arylalkyl substituent; and
(b) a fluorescent molecule.

2. The phospholipid dye of claim 1, wherein X is selected from the group of radioactive halogen isotopes consisting of ¹⁸F, ³⁶Cl, ⁷⁶Br, ⁷⁷Br, ⁸²Br, ¹²²I, ¹²³I, ¹²⁴I, ¹²⁵I, ¹³¹I and ²¹¹At.

3. The phospholipid dye of claim 1, wherein the phospholipid compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive isotope.

4. The phospholipid dye of claim 1, wherein said dye is selected from the group consisting of 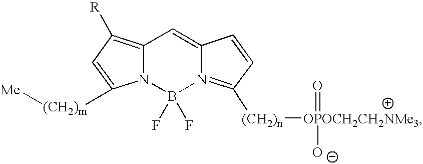
wherein n is an Integer 4 through 21 and m is an Integer 0 through 17; 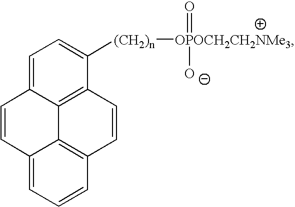
wherein n is an integer 4 through 22; 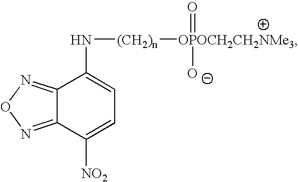
wherein n is an integer 4 through 22; 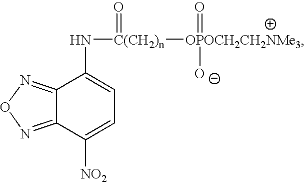
wherein n is an integer 4 through 21; 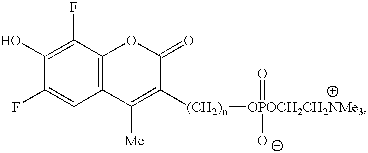
wherein n is an integer 4 through 22; 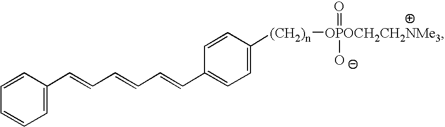
wherein n is an integer 3 through 8; and 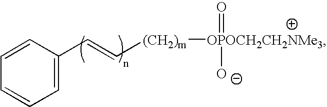
wherein n is an integer 4 or 5 and m is an integer 4 through 14.

5. The phospholipid dye of claim 1, wherein the fluorescent molecule exhibits fluorescence at a wavelength of about 300 nm to about 1000 nm.

6. A method for distinguishing a benign structure from a neoplastic tissue in a selected region by using an endoscope have at least two wavelength in a subject comprising the steps of:
(a) administering a fluorescently labeled tumor-specific agent to the subject;
(b) using a first technique to produce a visualization of the anatomy of the selected region using the first wavelength of an endoscope;
(c) using a second technique to produce a visualization of the distribution of fluorescence produced by the fluorescently labeled tumor-specific agent; and
(d) comparing the visualization of the anatomy of the selected region by the first wavelength to the visualization of the distribution of fluorescence by the second wavelength produced by the fluorescently labeled tumor-specific agent thereby distinguishing a benign structure from neoplastic tissue.

7. The method of claim 6, wherein the selected region is the gastro-intestinal tract and the respiratory tract.

8. The method of claim 6, wherein the first wavelength is about 400 nm to about 800 nm.

9. The method of claim 6, wherein the second wavelength is about 300 nm to 1000 nm.

10. The method of claim 6, wherein the fluorescently labeled tumor selective compound is a phospholipid dye, comprising of (a) a phospholipid compound of formula I or II 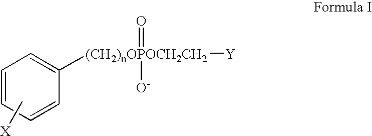
where X is a halogen; n is an integer between 8 and 30; and Y is selected from the group comprising NH₂, NR₂, and NR₃, wherein R is an alkyl or arylalkyl substituent or 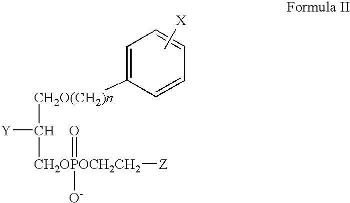
where X is a halogen; n is an integer between 8 and 30; Y is selected from the group consisting of H, OH, COOH, COOR and OR, and Z is selected from the group consisting of NH₂, NR₂, and NR₃, wherein R is an alkyl or arylalkyl substituent; and
(b) a fluorescent molecule.

11. The method of claim 10, wherein X is selected from the group of radioactive halogen isotopes consisting of ¹⁸F, ³⁶Cl, ⁷⁶Br, ⁷⁷Br, ⁸²Br, ¹²²I, ¹²³I, ¹²⁴I, ¹²⁵I, ¹³¹I and ²¹¹At.

12. The method of claim 10, wherein the phospholipid compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive isotope.

13. The method of claim 10, wherein said dye is selected from the group consisting of 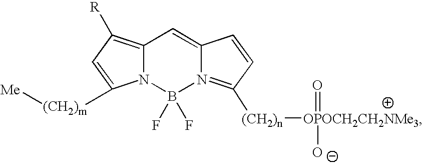
wherein n is an Integer 4 through 21 and m is an integer 0 through 17; 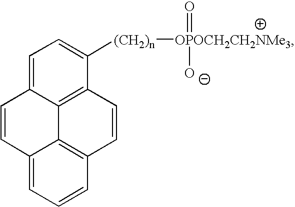
wherein n is an integer 4 through 22; 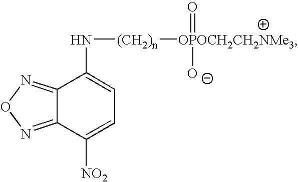
wherein n is an integer 4 through 22; 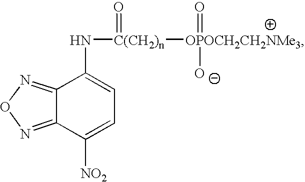
wherein n is an integer 4 through 21; 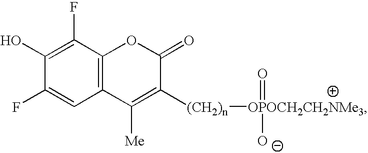
wherein n is an integer 4 through 22; 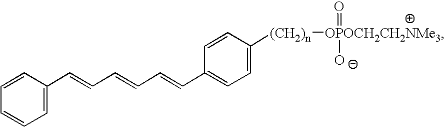
wherein n is an integer 3 through 8; and 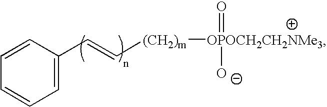
wherein n is an integer 4 or 5 and m is an integer 4 through 14.

14. The method of claim 9, wherein the fluorescent molecule exhibits fluorescence at a wavelength of about 300 nm to about 1000 nm.

15. A method of optimizing therapy treatment in a subject, comprising the steps of:
(a) providing a radiolabeled phospholipid compound wherein said compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is In the form of a radioactive Isotope, in a quantity of about 1 millicurie to about 100 millicurie;
(b) visualizing neoplastic tissue via SPECT or PET imaging;
(c) assessing therapy dosage to the subject by quantifying the distribution of the neoplastic tissue.

16. A method of monitoring tumor therapy response In a subject or effectiveness of a treatment methodology in a subject receiving the treatment for neoplasia, comprising the steps of:
(a) providing a radiolabeled phospholipid compound to the subject prior to treatment of neoplasia wherein said compound is 18-(p-Iodophenyl)octadecyl phosphocholine, 1-O-[18-(p-Iodophenyl)octadecyl]-1,3-propanediol-3-phosphocholine, or 1-O-[18-(p-Iodophenyl)octadecyl]-2-O-methyl-rac-glycero-3-phosphocholine, wherein iodine is in the form of a radioactive Isotope, in a quantity of about 1 millicurie to about 100 millicurie;
(b) providing the radiolabeled phospholipid compound to the subject of step (a), after the treatment of neoplasia in a quantity of about 1 millicurie to about 100 millicurie; and
(c) assessing difference in accumulation of the phospholipid compound from the pre-treatment of step (a) and the post-treatment of step (b) to determine the response in a subject or effectiveness of the treatment methodology, wherein a greater accumulation of the phospholipid compound in step (a) versus lesser accumulation of phospholipid compound in step (b) indicates a positive response to the treatment in a subject or an effective treatment methodology.
